(19) United States
(12) Patent Application Publication
(10) Pub. No.: US 2024/0148481 A1
(43) Pub. Date: May 9, 2024

(54) Method Of Producing A Multicolor Glass-Ceramic Blank For Dental Purposes, Multicolor Glass-Ceramic Blank, And Use Thereof

(71) Applicant: Ivoclar Vivadent AG, Schaan (LI)

(72) Inventors: Lars ARNOLD, Sevelen (CH); Walter ENTNER, Rankweil (AT)

(21) Appl. No.: 18/502,685

(22) Filed: Nov. 6, 2023

(30) Foreign Application Priority Data
Nov. 8, 2022 (EP) 22206185.5

(51) Int. Cl. A61C 13/00; B28B 3/02

(52) U.S. Cl. CPC A61C 13/0022; B28B 3/025; A61C 2201/002

(57) ABSTRACT

A method of producing a multicolor glass-ceramic blank (10) for dental purposes. A glass-ceramic blank (10) is produced from at least a first material powder (18) and a second material powder (20), wherein the first material powder (18) and the second material powder (20) are different-colored and wherein at least one of first material powder (18) and second material powder (20) has nanoparticles (14) and/or glass-ceramic particles (16). The first material powder (18) and the second material powder (20) are introduced into a mold (22) in order to form at least one powder mixture aggregate (26). Additionally, the powder mixture aggregate (26) is compressed by hot pressing in order to form the glass-ceramic blank (10). A multicolor glass-ceramic blank (10) is obtainable by such a method and the multicolor glass-ceramic blank (10) is used as dental material.

### CROSS-REFERENCE TO RELATED APPLICATIONS

This application claims priority to European patent application No. 22206185.5 filed on Nov. 8, 2022, the disclosure of which is incorporated herein by reference in its entirety.

### TECHNICAL FIELD

The invention relates to a method of producing a multicolor glass-ceramic blank for dental purposes. The invention is further directed to a multicolor glass-ceramic blank obtainable by such a method. The invention additionally relates to the use of such a multicolor glass-ceramic blank as dental material.

### BACKGROUND

In this connection, the use of glass-ceramic blanks in dental technology is known. Multicolor glass-ceramic blanks have the advantage that they can give a very good simulation of the visual properties of natural tooth material, especially by comparison with monochrome glass-ceramic blanks. This means that multicolor glass-ceramic blanks are particularly suitable for the production of esthetically demanding dental restorations having very good optical and mechanical properties. The optical properties relate not just to the color but also to the translucence of the dental restoration.

US 20200317561 is directed to a multi-colored glass ceramic blank and is hereby incorporated by reference in its entirety.

### SUMMARY

It is an object of the invention to further improve known methods of producing multicolor glass-ceramic blanks. What is to be specified is more particularly a method by which, in a simple manner, a multicolor glass-ceramic blank can be produced, with which a very good simulation of the optical properties of natural tooth material is possible, to which the shape of the ultimately desired dental restoration can be imparted by machine in a simple manner, and which, after the shaping, can be transformed to a dental restoration having excellent mechanical and optical properties.

The object is achieved by a method of producing a multicolor glass-ceramic blank for dental purposes. The multicolor glass-ceramic blank is produced from at least a first material powder and a second material powder, wherein the first material powder and the second material powder are different-colored. Moreover, at least one of first material powder and second material powder comprises nanoparticles and/or glass-ceramic particles. The method comprises:
introducing the first material powder and the second material powder into a mold, in order to form at least one powder mixture aggregate, and
compressing the powder mixture aggregate by hot pressing in order to form the glass-ceramic blank.

The method of the invention includes multiple variants.

In a first variant of the method of the invention, the first material powder and the second material powder are each introduced into the mold in dry form. Optionally, the first material powder and/or the second material powder forms a pelletized material. The first material powder and the second material powder are metered into the mold here in such a way as to result in a defined, continuous progression of the optical properties, especially a continuous color progression, within the mold. In this variant, it is additionally possible to process the powder mixture aggregate formed by the first material powder and the second material powder directly by hot pressing to give the glass-ceramic blank. Optionally, the powder mixture aggregate is processed via the intermediate step of a heat treatment to give the glass-ceramic blank. In the intermediate step of heat treatment, it is possible to thermally remove at least one constituent of the powder mixture aggregate which is unwanted in the glass-ceramic blank. For example, in this connection, binders are baked out by the heat treatment.

In a second variant of the method of the invention, the first material powder and/or the second material powder are introduced into the mold as a suspension. The suspension is especially an aqueous suspension of the first material powder and/or an aqueous suspension of the second material powder. In this variant, the powder mixture aggregate must always be dried before being processed further to give the glass-ceramic blank. A drying step is thus conducted. The dried powder mixture aggregate can then be processed further directly to give the glass-ceramic blank. As already elucidated in connection with the first variant, it is possible in the second variant too to provide an intermediate step for production of a green body and/or an intermediate step for production of a white body.

In a third variant, the first material powder and the second material powder are introduced into the mold in the form of a green body comprising the first material powder and the second material powder. The green body is obtained here from a preliminary process. In this connection, the powder mixture aggregate is formed by the green body.

In a fourth variant, the first material powder and the second material powder are introduced into the mold in the form of a white body comprising the first material powder and the second material powder. The white body is obtained here from a preliminary process. In this variant, the powder mixture aggregate is formed by the white body.

In all variants, the first material powder and/or the second material powder comprises nanoparticles and/or glass-ceramic particles. This in turn embraces nine variants. In a variant A, the first material powder comprises nanoparticles. In a variant B, the second material powder comprises nanoparticles. In a variant C, both the first material powder and the second material powder comprise nanoparticles. In a variant D, the first material powder comprises glass-ceramic particles. In a variant E, the second material powder comprises glass-ceramic particles. In a variant F, both the first material powder and the second material powder comprise glass-ceramic particles. In a variant G, the first material powder comprises both nanoparticles and glass-ceramic particles. In a variant H, the second material powder comprises both nanoparticles and glass-ceramic particles. In a variant I, both the first material powder and the second material powder comprise both nanoparticles and glass-ceramic particles.

By contrast with the prior art, in none of the above variants A to I do both the first material powder and the second material powder consist of glass powder. It is of course not ruled out here that the first material powder and/or the second material powder also comprises glass powder. The use of glass-ceramic particles and/or nanoparticles has the effect that the production of the glass-ceramic blank is simplified. Since glass-ceramic particles already comprise nucleated and/or crystallized sections, these are stable up to a certain temperature, even under pressure. Premature compaction can thus be ruled out. In this way, unwanted inclusions of gas are prevented. With regard to the nanoparticles, these offer a comparatively high surface area in a defined volume. This is beneficial to surface crystallization. It is thus possible to create crystal structures comparatively quickly. The glass-ceramic particles and/or the nanoparticles are thus chosen such that they facilitate or actually enable the production process, since they serve as nucleators for the later crystallization of other phases. Alternatively or additionally, the glass-ceramic particles and/or the nanoparticles may be chosen such that they have a positive effect on the optical and/or mechanical properties of the glass-ceramic blank. In particular, the glass-ceramic particles and/or the nanoparticles may be used to bring about a desired color progression and/or a desired translucence progression in the glass-ceramic blank. In addition, by means of the glass-ceramic particles and/or the nanoparticles, it is possible to create a desired opalescent progression and/or a desired fluorescence progression. For this purpose, the glass-ceramic particles and/or the nanoparticles must have appropriate opalescent properties and/or appropriate fluorescence properties. In this way, it is possible to give a particularly good simulation of the optical properties of natural teeth.

Nanoparticles in the present context are understood to mean particles having a particle size of 1 nm to 100 nm.

In the context of the present invention, glass-ceramic particles are understood to mean that both particles of nucleated glass-ceramic and particles of crystallized glass-ceramic are included.

The glass-ceramic particles have a grain size of more than 100 nm and less than 2 mm.

In one embodiment, the first material powder and the second material powder are introduced into the mold in locally differing mixing ratios, so as to result in a color progression in the glass-ceramic blank. It is possible in this way to easily and reliably create a continuous color progression in the glass-ceramic blank. The color progression may also be locally different within the glass-ceramic blank. Because of the fact that powders are introduced into the mold, the color progression can be very finely adjusted.

In one variant, the powder mixture aggregate is heat-treated. This can especially serve to thermally remove a constituent of the powder mixture aggregate which is unwanted in the glass-ceramic blank. This is also referred to as baking. For example, it is possible by means of such a heat treatment to remove binders from the powder mixture aggregate. In addition, the heat treatment can bring about preliminary sintering.

In one alternative, at least one of first material powder and second material powder comprises rounded glass particles, rounded nanoparticles and/or rounded glass-ceramic particles. In this connection, the rounding improves the free flow of the glass particles, nanoparticles and/or glass-ceramic particles. This is especially true by comparison with non-rounded particles. The rounding facilitates the introducing of the first material powder and the second material powder into the mold. This is particularly helpful when the first material powder and the second material powder are being introduced directly into the mold. It is thus possible to dispense with preliminary processes for production of a green body and/or a white body. This simplifies the production method for production of the glass-ceramic blank.

The glass particles, nanoparticles and/or glass-ceramic particles may be rounded mechanically, for example. In this connection, the glass particles, nanoparticles and/or glass-ceramic particles are ground using grinding bodies. Alternatively or additionally, the glass particles, nanoparticles and/or glass-ceramic particles may be rounded thermally. For this purpose, the glass particles, nanoparticles and/or glass-ceramic particles may be subjected to a plasma treatment.

In the context of present invention, glass particles used may be material systems for formation of glass-ceramics via the melt casting method that are suitable for the formation of lithium disilicate and/or lithium metasilicate.

Glass-ceramic particles used may be already crystallized compositions from the group of the aforementioned glass particles at different crystallization stages. These may optionally include pigments, for example fluorescence pigments such as europium-doped strontium aluminate. Europium-doped strontium aluminate is described in EP3696150A1 and corresponding U.S. Pat. No. 11,440,834 B2, which U.S. patent is hereby incorporated by reference.

In addition, in the context of the present invention, nanoparticles may be pigments or opacifiers.

In addition, it is possible that nanoparticles include ceramic components for adjustment of processing properties as defects in the microstructure. Examples of these are zirconium dioxide, aluminum oxide and silicon dioxide in various modifications.

It is also possible for nanoparticles to take the form of nucleating agents for heterogeneous nucleation (e.g. metal colloids, other extrinsic crystals).

It is further possible for nanoparticles used to be particularly small particles from the groups of the glass particles or glass-ceramic particles. This serves to increase sintering activity.

In the compression of the powder mixture aggregate by hot pressing, the powder mixture aggregate may be positioned in the mold. This means that the hot pressing takes place in that mold into which the first material powder and the second material powder have been introduced to form the powder mixture aggregate. Thus, the method of the invention is executed using merely a single mold into which the first material powder and the second material powder are introduced and which is used in the hot pressing. The method can therefore proceed in a particularly efficient manner.

In the compressing of the powder mixture aggregate by hot pressing, the powder mixture aggregate can first be heated to a temperature of at least 700° C. and then a compression force can be applied. The heating of the powder mixture aggregate to 700° C. achieves the effect that the powder mixture aggregate is reliably degassed. Only thereafter is the compression force applied. In this way, it is possible to produce high-quality glass-ceramic blanks. This is true especially when the first material powder and the second material powder are introduced directly into the mold and intermediate steps for formation of a green body and/or a white body are omitted.

The attainment of the temperature of at least 700° C. can be used as trigger criterion for the applying of the compression force. The powder mixture aggregate is thus monitored with regard to its temperature. This is based on the finding that, on attainment of a temperature of 700° C., the powder mixture aggregate is sufficiently degas sed. The method overall and the compacting by hot pressing in particular can thus be controlled easily and reliably.

The powder mixture aggregate is preferably compressed by hot pressing to a density of at least 99.9% of the base material of the first material powder and/or of the base material of the second material powder. In other words, the glass-ceramic blank after the compression by hot pressing has a density corresponding essentially to the density of the base material of the first material powder and/or of the base material of the second material powder. In this way, it is possible to produce glass-ceramic blanks of high mechanical quality.

In one embodiment, the first material powder and the second material powder are introduced into the mold at least two separate sites in the mold, in order to form at least two powder mixture aggregates. With such a mold, it is consequently possible to produce at least two glass-ceramic blanks, one from each powder mixture aggregate. Such a mold may also be referred to as a multiple mold since it is possible to simultaneously produce two or more glass-ceramic blanks using such a mold. In this way, it is possible to produce a comparatively large number of glass-ceramic blanks within a comparatively short time.

In general terms, the method of the invention can thus be performed in conjunction with a single mold, i.e. a mold designed to produce a single glass-ceramic blank, or in conjunction with a multiple mold.

The compressing of the powder mixture aggregate by hot pressing can be effected at a temperature of 650° C. to 980° C., in particular 700° C. to 750° C., and at a pressure of in particular MPa to 50 MPa, preferably 10 MPa to 30 MPa. What are formed are thus glass-ceramic blanks of high mechanical and optical quality. At the same time, there is avoidance of temperatures that are too low in material-specific terms and hence either do not permit compaction or lead to unwanted inclusions of gas. Excessively high temperatures in material-specific terms are likewise avoided, which can lead to formation of unwanted crystals.

The compacting of the powder mixture aggregate by hot pressing can be effected for a period of 0.1 minute to 10 minutes, preferably 0.3 minute to 5 minutes. The compacting by hot pressing thus takes only a comparatively short period of time. It is thus possible to produce the glass-ceramic blank within a comparatively short time. It will be apparent that the mechanical and optical demands on the glass-ceramic blank are simultaneously met.

The compacting of the powder mixture aggregate by hot pressing can be effected at an atmospheric pressure of less than 0.1 bar and preferably of 0.01 bar to 0.08 bar. In simplified terms, the compacting of the powder mixture aggregate by hot pressing takes place under reduced pressure. In this way, unwanted reactions with an ambient atmosphere are reduced or ruled out. In this way, the molds are also protected.

In one variant, the glass-ceramic blank is a multiple blank. This means that the glass-ceramic blank is designed for use for two or more dental restorations. This implies that the glass-ceramic blank has to be divided into at least two pieces before, during or after the creation of the dental restorations. For example, the glass-ceramic blank can be cut or broken up. This preferably precedes the creation of the dental restoration. The blank can also be separated within a milling or grinding machine during or after the creation of the dental restoration. In this case, the separation involves removal of material. By the producing of multiple blanks, it is thus possible in an efficient and reliable manner to produce many glass-ceramic blanks within a short time.

The method may also comprise opening of the mold and removal of the glass-ceramic blank. In particular, the mold is opened while both the mold and the glass-ceramic blank are still hot. In this connection, reference is also made to hot demolding or to demolding in the hot state. In other words, the mold and the glass-ceramic blank present therein are not cooled in a defined manner before the mold is opened and the glass-ceramic blank is removed. In this way, the efficiency of utilization of the mold is increased. It will be apparent that the glass-ceramic blank then cools down outside the mold.

The object is also achieved by a multicolor glass-ceramic blank obtainable by a method of the invention. As already elucidated, such a glass-ceramic blank can be produced efficiently, which especially includes cost-efficiency. The glass-ceramic blank is of high quality from mechanical and optical aspects. The glass-ceramic blank can thus be processed further to give a dental restoration. In particular, such a blank can be used to create crowns, abutments, abutment crowns, inlays, onlays, veneers, bridges and overdentures. Because of the good mechanical and optical properties of the glass-ceramic blank, the dental restorations also have good mechanical and optical properties.

The object is also achieved by use of the multicolor glass-ceramic blank of the invention as dental material. In particular, the multicolor glass-ceramic blank of the invention is used to create a dental restoration. Because of the good mechanical and optical properties of the glass-ceramic blank, it is possible in this way to create dental restorations having good mechanical and optical properties.

### BRIEF DESCRIPTION OF THE DRAWINGS

The invention is elucidated hereinafter by various working examples that are shown in the appended drawings. The figures show:

FIG. 1 a multicolor glass-ceramic blank of the invention that has been produced by a method of the invention, and the use thereof for creation of a dental restoration,

FIG. 2 a sequence of a method of the invention for production of the multicolor glass-ceramic blank from FIG. 1,

FIG. 3 a variant of the method from FIG. 2, and

FIG. 4 a further variant of the method from FIG. 2.

### DETAILED DESCRIPTION

FIG. 1 shows a multicolor glass-ceramic blank 10.

The glass-ceramic blank 10 comprises a main phase 12 of glass-ceramic, with both nanoparticles 14 and glass-ceramic particles 16 embedded in this main phase 12 of glass-ceramic.

In the diagram in FIG. 1, the nanoparticles 14 and the glass-ceramic particles 16 are illustrated merely symbolically and in excessive size.

The glass-ceramic particles 16 differ from the main phase 12 of glass-ceramic in that the glass-ceramic particles 16 were already present in the production of the main phase 12. This will be elucidated in detail later on.

The glass-ceramic blank 10, i.e. the main phase 12 in particular, has been produced from different-colored material powders, such that the glass-ceramic blank 10 has a continuous color progression. This is illustrated in FIG. 1 by the hatching with varying width.

The glass-ceramic blank 10 is used as dental material for creation of a dental restoration R.

In this connection, the glass-ceramic blank 10 is processed with removal of material in step (a), such that the glass-ceramic blank 10 takes on the shape of the dental restoration R to be created. It will be apparent that the dental restoration in FIG. 1 is merely a schematic illustration.

In a step (b) that follows step (a), the glass-ceramic blank 10 that has been processed with removal of material is thermally cured. In this connection, crystallization processes take place within the glass-ceramic blank 10. The mechanical properties of the glass-ceramic blank 10 are thus established.

Subsequently, the dental restoration R created in this way can be used in a patient.

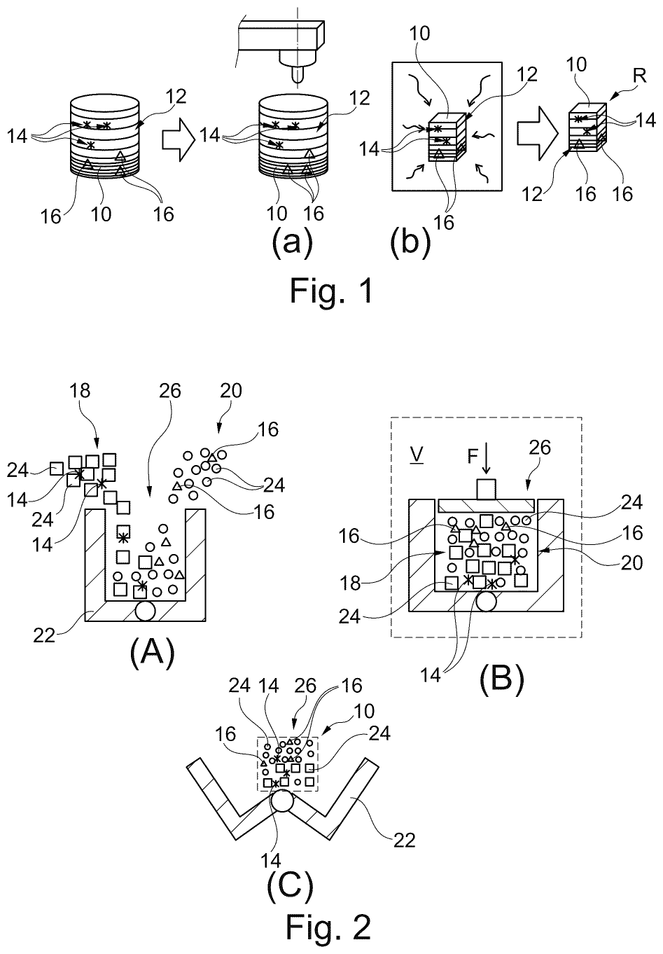

The glass-ceramic blank 10 is produced by a method which is elucidated hereinafter by FIGS. 2, 3 and 4.

In the example according to FIG. 2, in a first method step A, a first material powder 18 and a second material powder 20 are introduced into a mold 22.

The first material powder 18 and the second material powder 20 are different-colored. In addition, the first material powder 18 comprises nanoparticles 14 and glass particles 24. The second material powder 20 comprises glass-ceramic particles 16 and glass particles 24. The glass particles 24, the nanoparticles 14 and the glass-ceramic particles 16 are rounded.

In addition, the first material powder 18 and the second material powder 20 are introduced into the mold 22 in locally different mixing ratios.

It will be apparent that, in FIG. 2, the glass particles 24, the nanoparticles 14 and the glass-ceramic particles 16 are illustrated merely schematically and in greatly enlarged form. In order to symbolize the different coloring, the glass particles 24 of the first material powder 18 are shown as squares and the glass particles 24 of the second material powder 20 as circles. For better clarity, only some particles are given a reference numeral.

In the working example shown, the first material powder 18 accounts for a greater share than the second material powder 20 in a lower region of the mold 22. The reverse is true in an upper region of the mold 22.

Since the first material powder 18 and the second material powder 20 are different-colored, this results in a color progression within the powder mixture aggregate 26, which is formed by the introducing of the first material powder 18 and the second material powder 20 into the mold 22. This color progression is maintained in the finished glass-ceramic blank 10.

The powder mixture aggregate 26 can optionally be compressed within the mold 22 to give a green body.

Further optionally, the powder mixture aggregate 26 may be heat-treated.

In a subsequent method step B, the powder mixture aggregate 26 is compressed by hot pressing. In this way, the glass-ceramic blank 10 is formed from the powder mixture aggregate 26.

For this purpose, the powder mixture aggregate 26 remains in the mold 22.

In detail, in the course of hot pressing, the powder mixture aggregate 26 is first heated to a temperature of 700° C. Only when the powder mixture aggregate 26 reaches the temperature of 700° C. is a compression force F applied to the powder mixture aggregate 26. In other words, the attaining of the temperature of 700° C. is the trigger criterion for the applying of the compression force F.

The compression force F is chosen such that the powder mixture aggregate 26 is subjected to a pressure of 10 MPa to 30 MPa. In the present context, the pressure is 20 MPa.

Moreover, the temperature of the powder mixture aggregate 26 is increased further during the applying of the compression force F. In the present context, the temperature is increased to 730° C.

Moreover, the compression takes place in a vacuum chamber V. The pressure within the vacuum chamber V is from 0.01 to 0.08 bar.

In the present working example, the pressure mentioned and the temperature mentioned are maintained for a period of 4 minutes.

Thereafter, in a method step C, the mold 22 is opened and the glass-ceramic blank 10 is removed from the mold 22. This is done essentially directly after method step B. There is thus no defined cooling operation.

It will be apparent that the opening of the mold 22 which is shown in method step C is merely schematic, and the mold 22 can be opened in any other suitable manner.

The glass-ceramic blank 10 produced in this way achieves a density of 99.9% of the base material of the first material powder 18.

With regard to the geometric dimensions of the glass-ceramic blank 10, the dimensions thereof are such that, by means of a method already elucidated in association with FIG. 1, a single dental restoration R can be elaborated from the glass-ceramic blank 10.

This glass-ceramic blank 10 may take the form of a crown blank, inlay blank or bridge blank. Such blanks and corresponding dimensions are common knowledge.

For example, a crown blank has dimensions of 18.4 mm×14.7 mm×12.5 mm. A bridge blank may have dimensions of 15 mm×32 mm×15 mm.

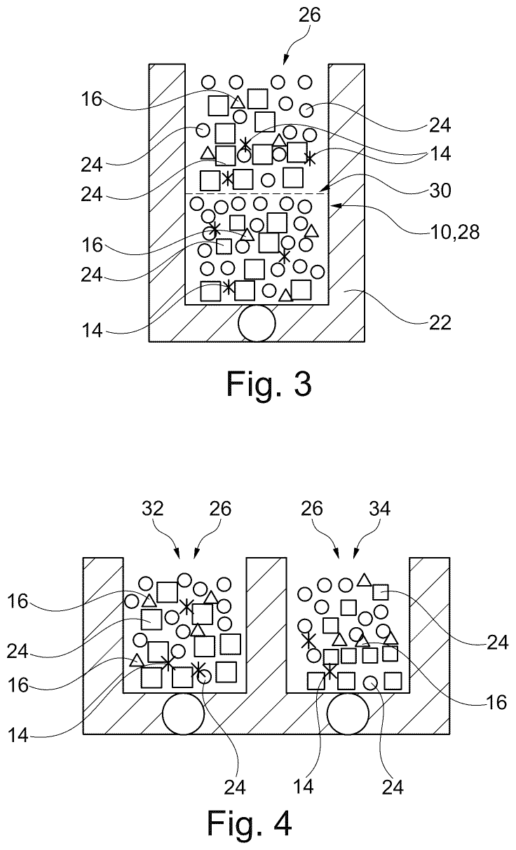

FIG. 3 illustrates one variant of the method shown in FIG. 2.

All that are addressed here are the differences with respect to the method from FIG. 2. Identical or mutually corresponding elements are given the same reference numerals.

In the variant according to FIG. 3, a mold 22 is used, the interior of which is essentially twice as large as in the case of the mold 22 which is used in the method according to FIG. 2.

It is thus possible in the variant according to FIG. 3 to produce a glass-ceramic blank 10 at least twice as large as the glass-ceramic blank 10 that can be produced by the method from FIG. 2.

Accordingly, the dimensions of the glass-ceramic blank 10 produced by the method according to FIG. 3 are such that, by means of a method elucidated in association with FIG. 1, two dental restorations R can be elaborated from the glass-ceramic blank 10.

It will be apparent that glass-ceramic blanks 10 suitable for production of more than two dental restorations R are also conceivable.

The glass-ceramic blank 10 that can be produced by means of the variant from FIG. 3 can therefore also be referred to as multiple blank 28.

Within the multiple blank 28, a separation plane 30 may be provided. When the multiple blank 28 is divided along this separation plane 30, the result is two glass-ceramic blanks 10, the dimensions of which correspond to a glass-ceramic blank 10 obtainable by means of the method according to FIG. 2.

A further variant of the method of producing a glass-ceramic blank 10 is illustrated in FIG. 4.

Again, all that are addressed are the differences from the method according to FIG. 2. Identical or mutually corresponding elements are given the same reference numerals.

The differences again relate to the mold 22 used.

In the method according to FIG. 4, the mold 22 has two cavities 32, 34, and it is possible to accommodate a powder mixture aggregate 26 for production of a glass-ceramic blank 10 in each of the cavities 32, 34. In other words, by means of such a mold 22, it is possible to simultaneously produce two glass-ceramic blanks 10, the dimensions of which correspond to the glass-ceramic blank 10 obtainable by means of the method according to FIG. 2.

Accordingly, in the variant according to FIG. 4, in method step A, the first material powder 18 and the second material powder 20 are introduced into the mold 22 at at least two separate sites in the mold 22 that correspond to the cavities 32, 34. Thus, two separate powder mixture aggregates 26 are formed.

Such a mold 22 may also be referred to as multiple mold 36.

The above examples may also be combined. In this connection, one conceivable variant is, for example, one in which the method is performed with a multiple mold designed to produce two or more multiple blanks. In this way, it is possible to simultaneously produce a particularly large number of glass-ceramic blanks.

### LIST OF REFERENCE NUMERALS

10 glass-ceramic blank
12 main phase
14 nanoparticles
16 glass-ceramic particles
18 first material powder
20 second material powder
22 mold
24 glass particles
26 powder mixture aggregate
28 multiple blank
30 separation plane
32 cavity
34 cavity
36 multiple mold
F compression force
R dental restoration
V vacuum chamber

1. A method of producing a multicolored glass-ceramic blank (10) for dental purposes, comprising at least a first material powder (18) and a second material powder (20), wherein the first material powder (18) and the second material powder (20) are different-colored and wherein at least one of first material powder (18) and second material powder (20) comprises nanoparticles (14) and/or glass-ceramic particles (16), wherein the method comprises:
introducing the first material powder (18) and the second material powder (20) into a mold (22), in order to form at least one powder mixture aggregate (26), and
compressing the powder mixture aggregate (26) by hot pressing to form the glass-ceramic blank (10).

2. The method as claimed in claim 1, wherein the first material powder (18) and the second material powder (20) are introduced into the mold (22) in locally different mixing ratios, to result in a color progression in the glass-ceramic blank (10).

3. The method as claimed in claim 1, further comprising heat treatment of the powder mixture aggregate (26).

4. The method as claimed in claim 1, wherein at least one of the first material powder (18) and the second material powder (20) comprises rounded glass particles (24), rounded nanoparticles (14) and/or rounded glass-ceramic particles (16).

5. The method as claimed in claim 1, wherein, in the compressing of the powder mixture aggregate (26) by hot pressing, the powder mixture aggregate (26) is positioned in the mold (22).

6. The method as claimed in claim 1, wherein, in the compressing of the powder mixture aggregate (26) by hot pressing, the powder mixture aggregate (26) is first heated to a temperature of at least 700° C. and then a compression force (F) is applied.

7. The method as claimed in claim 6, wherein the attainment of the temperature of at least 700° C. is used as trigger criterion for the applying of the compression force (F).

8. The method as claimed in claim 1, wherein the powder mixture aggregate (26) is compressed by hot pressing to a density of at least 99.9% of a base material of the first material powder (18) and/or of a base material of the second material powder (20).

9. The method as claimed in claim 1, wherein the first material powder (18) and the second material powder (20) are introduced into the mold (22) at least two separate sites in the mold (22) in order to form at least two powder mixture aggregates (26).

10. The method as claimed in claim 1, wherein the powder mixture aggregate (26) is compressed by hot pressing at a temperature of 650° C. to 780° C. and at a pressure of 5 MPa to 50 MPa.

11. The method as claimed in claim 1, wherein the powder mixture aggregate (26) is compressed by hot pressing at a temperature of 700° C. to 750° C., and at a pressure of 10 MPa to 30 MPa.

12. The method as claimed in claim 1, wherein the powder mixture aggregate (26) is compressed by hot pressing for a duration of 0.1 minute to 10 minutes.

13. The method as claimed in claim 1, wherein the powder mixture aggregate (26) is compressed by hot pressing for a duration of 0.3 minute to 5 minutes.

14. The method as claimed in claim 1, wherein the powder mixture aggregate (26) is compressed by hot pressing at an atmospheric pressure of less than 0.1 bar.

15. The method as claimed in claim 1, wherein the powder mixture aggregate (26) is compressed by hot pressing at an atmospheric pressure of 0.01 bar to 0.08 bar.

16. The method as claimed in claim 1, wherein the glass-ceramic blank (10) is a multiple blank (28).

17. The method as claimed in claim 1, further comprising opening the mold (22) and removing the glass-ceramic blank (10).

18. A multicolor glass-ceramic blank (10) obtainable by the method as claimed in claim 1.

19. A method of using the multicolor glass-ceramic blank (10) as claimed in claim 18 as dental material or for production of a dental restoration (R).
